(19) United States
(12) Patent Application Publication
(10) Pub. No.: US 2025/0082786 A1
(43) Pub. Date: Mar. 13, 2025

(54) FLUORESCENT MARKERS FOR NEUROFIBRILLAR TANGLES AND USES THEREOF

(71) Applicants: FONDAZIONE ISTITUTO ITALIANO DI TECNOLOGIA, Genova (IT); Disruptive Technological Advances In Life Science S.r.l. - Società Benefit (D-TAILS S.r.l. SB), Roma (IT)

(72) Inventors: Alberto BOFFI, Roma (IT); Francesca GHIRGA, Genova (IT); Alessandro SOLOPERTO, Genova (IT); Silvia DI ANGELANTONIO, Roma (IT)

(73) Assignees: FONDAZIONE ISTITUTO ITALIANO DI TECNOLOGIA, Genova (IT); Disruptive Technological Advances In Life Science S.r.l. - Società Benefit (D-TAILS S.r.l. SB), Roma (IT)

(21) Appl. No.: 18/551,485

(22) PCT Filed: Apr. 20, 2022
(86) PCT No.: PCT/IB2022/053673

§ 371 Date: Sep. 20, 2023

(30) Foreign Application Priority Data
Apr. 23, 2021 (IT) 102021000010382

(51) Int. Cl. A61K 49/00; C07F 5/02

(52) U.S. Cl. CPC A61K 49/0021; C07F 5/022

(57) ABSTRACT

The present invention relates to new fluorescent markers selectively binding tau protein, uses thereof, methods for imaging the neurofibrillary tangles of the tau protein in the retina of a subject, as well as a device that enables the implementation of said methods.

The present invention relates to new fluorescent markers selectively binding the neurofibrillary tangles of the tau protein, a composition comprising said markers, uses thereof, methods for imaging the neurofibrillary tangles of the tau protein in the retina of a subject, as well as a device that enables the implementation of said methods.

### STATE OF THE ART

Alzheimer's disease (AD) is a neurodegenerative disease responsible for more than 80% of cases of senile dementia. The limited scientific evidence underlying the pathogenic mechanisms of AD has made it difficult to develop targeted and effective therapies and diagnoses and, to date, no solutions have been found that guarantee an improvement in the life of high-risk patients or effective prevention that allows to reduce health expenditure. [Graham V. W. et al., 2017] Despite numerous studies to elucidate the pathogenic mechanisms and efforts of the pharmaceutical industry, there is still no effective therapy to treat AD or significantly block the progression of symptoms. [Masters C. L et al., 2015; Graham V. W. et al, 2017] Consequently, in order to develop a therapeutic strategy capable of preventing AD, research has focused on the study of accurate, preferably early, and specific diagnostic methods for this disease. Numerous advances have been made following the introduction of cerebrospinal fluid analysis (CSF) [Olsson B. et al., 2016] and diagnostic neuroimaging techniques [Pietrzak K. et al., 2018].

These techniques allow to study the presence of functional alterations in the brain and biomarkers associated with AD.

These techniques, however, despite being relatively specific and selective, have numerous limitations (very expensive, invasive, they provide a positive result appreciable only when cognitive disorders are already advanced) and therefore cannot be used as the sole diagnostic tool but must be associated to postmortem evaluation of the pathophysiological traits.

Three main biomarkers have been identified for AD:
the Aβ42 peptide, which is the main component of the β-amyloid plaques,
the coupled units of hyperphosphorylated protein (P-Tau) and
the total-Tau monomers (t-Tau) which are the key brick of the neurofibrillary tangles (NFTs).

Recently, it has been shown that the presence and quantity of β-amyloid plaques in patients with dementia cannot be entirely related to the progression and clinical manifestations of the disease itself. On the contrary, numerous studies have shown a more rigorous correlation between the number of neurofibrillary tangles of the Tau protein and disease progression.

In this regard, although several fluorescent probes for the identification of AD biomarkers have been described in the literature and/or have been patented, only a limited number of these were highly selective in vivo for the aggregates of the Tau protein and to date, Tau-specific fluorophores are not commercially available. Therefore, the need is strongly felt to identify a new class of fluorophores capable of binding the NFTs of the Tau protein in a highly selective way.

### SUMMARY OF THE INVENTION

The present invention provides a new fluorescent marker selectively binding tau protein of formula I:

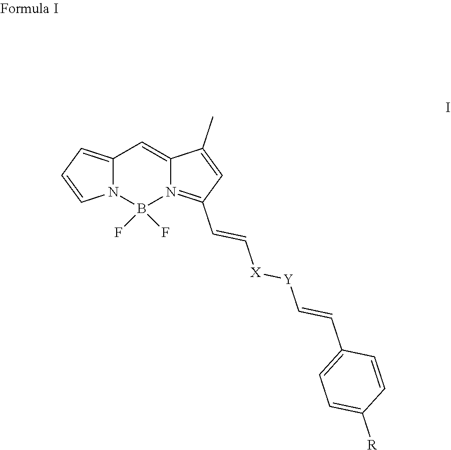

wherein X and Y are carbon atoms linked either by a double bond with E or Z configuration or by an aromatic or heteroaromatic para-substituted ring, or by an aromatic or heteroaromatic 1,4 disubstituted ring;
R is hydrogen, halogen, NH(RA), N(RA)₂, NHC(═O)RA, ORA, OC(═O)RA, SRA, SO₂RA, SO₃RA, OSO₂RA, OSO₃RA, C(RA)₃, or C₅₋₇ aromatic or aliphatic heterocycle; and
in each substituent, RA is: hydrogen; halogen; hydroxyl; CF₃, a C₁₋₇ saturated or unsaturated chain, linear or branched containing up to three independent heteroatoms selected among nitrogen oxygen and sulphur; C₅₋₇ cycloalkyl; phenyl; C₅₋₇ heterocycle or n-ethyleneglycol.

As clearly evident from the data reported in the experimental section of the present specification, the inventors have found that fluorescent markers of formula I, particularly the fluorescent compounds herein defined as BT1-BT4 and BT6, have a high binding affinity for the 6-mer model of the PHF6 fragment, which is present in the R3 region of the microtubule-binding tau protein and responsible for the propensity of the protein itself to assemble into fibrils.

The results obtained by the in vitro assays illustrated below clearly indicate said fluorescent compounds as new effective markers capable of selectively binding the neurofibrillary aggregates of the tau protein.

The present invention further provides a method for the preparation of a fluorescent marker of formula I as well as imaging methods using said marker.

Advantageously, the fluorescent markers of the invention demonstrate efficient penetration of the blood-brain barrier and capability to bind the neurofibrillary tangles of the Tau protein within the retina. As neurofibrillary tau tangles are associated to tauopathies, the detection of neurofibrillary tangles in the retina of a subject can be used as relevant information in the diagnosis or to assist in the diagnosis of tauopathies.

At present, most of the conventional methods to detect tau neurofibrillary tangles are based on the post-mortem analysis of brain tissues. One major challenge to early diagnosis of tauopathies is represented by the complexity of conventional diagnostic methods as these generally involve not only the immunohistochemical demonstration of abnormal tau deposition in the brain, but also the detection of the presence or absence and amount of other non-tau proteins in the brain, as well as the study of the morphological characteristics of the tau in different regions of the brain.

Advantageously, thanks to their high affinity and specificity for the neurofibrillary tangles of the Tau protein, together with their capability to cross the blood-brain barrier, the fluorescent markers of the present invention can be used to provide new imaging methods providing images that can be subsequently analysed by a specialist in order to obtain information allowing an effective, non-invasive, early diagnosis of tauopathies or that can be used in the assessment of the effectiveness of a medical treatment of a tauopathy and/or of the progression of a tauopathy.

Therefore, the subjects of the present invention are:
A fluorescent marker of formula I:

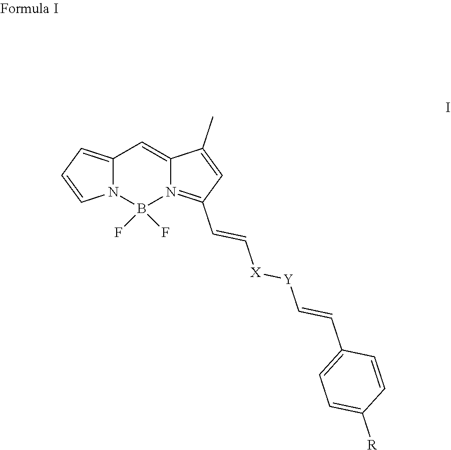

wherein X and Y are carbon atoms linked either by a double bond with E or Z configuration or by an aromatic or heteroaromatic para-substituted ring, or by an aromatic or heteroaromatic 1,4 disubstituted ring;
   R is hydrogen, halogen, NH(RA), N(RA)₂, NHC(═O)RA, ORA, OC(═O)RA, SRA, SO₂RA, SO₃RA, OSO₂RA, OSO₃RA, C(RA)₃, or C₅₋₇ aromatic or aliphatic heterocycle; and
in each substituent, RA is: hydrogen; halogen; hydroxyl; CF₃; a C₁₋₇ saturated or unsaturated chain, linear or branched containing up to three independent heteroatoms selected among nitrogen oxygen and sulphur; C₅₋₇ cycloalkyl; phenyl; C₅₋₇ heterocycle or n-ethyleneglycol, wherein said marker selectively binds Tau protein neurofibrillary tangles.
A method for the preparation of a fluorescent marker of formula I comprising the following steps:
i. subjecting 4,4-Difluoro-1,3-dimethyl-4-bora-3a,4a-diaza-s-indacene to Knoevenagel condensation reaction with an aldehyde of formula II,

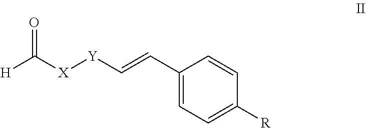

wherein X and Y are carbon atoms linked either by a double bond with E or Z configuration or by an aromatic or heteroaromatic para-substituted ring, or by an aromatic or heteroaromatic 1,4 disubstituted ring;
R is hydrogen, halogen, NH(RA), N(RA)₂, NHC(═O)RA, ORA, OC(═O)RA, SRA, SO₂RA, SO₃RA, OSO₂RA, OSO₃RA, C(RA)₃, or C₅₋₇ aromatic or aliphatic heterocycle; and in each substituent, RA is: hydrogen; halogen, hydroxyl, CF₃, a C₁₋₇ saturated or unsaturated chain, linear or branched containing up to three independent heteroatoms selected among nitrogen oxygen and sulphur; C₅₋₇ cycloalkyl; phenyl; C₅₋₇ heterocycle or n-ethyleneglycol;
ii. subjecting the solution obtained in step i. to liquid-liquid extraction (LLE) followed by a purification step to provide said fluorescent marker of formula I.
A composition comprising a fluorescent marker of formula I and one or more additional excipients and/or carriers.
A fluorescent marker of formula I or the composition comprising a marker of formula I as defined in the present description for use in the detection of neurofibrillary tangles of the Tau protein.
A method for the detection of neurofibrillary tangles of the Tau protein comprising the steps of:
   contacting a fluorescent marker of formula I or a composition comprising a fluorescent marker of formula I with a biological sample under conditions wherein said fluorescent marker binds to the neurofibrillary tangles of the tau protein;
   detecting the fluorescent marker of formula I bound to said biological sample.
An imaging method comprising the steps of
   administering to a subject a fluorescent marker of formula I or a composition comprising a fluorescent marker of formula I as described in the present specification,
   carrying out a non-invasive fluorescence imaging of the retina of said subject, wherein the detection of fluorescence from said marker of formula I indicates the binding of said marker to the retina.
A method for determining a fluorescence graph comprising the steps of
   administering to a subject a fluorescent marker of formula 1 or a composition comprising a fluorescent marker of formula 1 and carrying out a non-invasive quantitative fluorescence imaging on the retina of said subject, at a plurality of successive time instants tᵢ, where i is 0 to n, so obtaining corresponding fluorescence values;
using the obtained fluorescence values for determining said fluorescence graph.
A computer program for monitoring the progression of the amount of neurofibrillary tangles of tau protein in a subject's retina, including a list of instructions which, when performed on an electronic computer, provided a first fluorescence value f₀ at a time instant t₀ and one or more fluorescence values fₙ at one or more time instants tₙ, wherein n is an integer greater than 0 increasing progressively at each subsequent time instant tₙ, and in which each tₙ corresponds to subsequent instants of time following t₀, said values being obtained with any of the methods as defined in the present specification, implements the following steps:
   comparing, progressively from f₀ to fₙ, each of said values, and
   detecting a decrease in the amount of said neurofibrillary tangles when each fₙ fluorescence value is lower than f₀ and lower than the preceding fₙ,
   detecting an increase in the amount of said neurofibrillary tangles when each fₙ fluorescence value is higher than f₀ and higher than the preceding fₙ,
   detecting a substantial absence of variation in the amount of said neurofibrillary tangles when each fₙ fluorescence value is the same or substantially the same than f₀ and than the preceding fₙ.
A device for the automatic measurement of the fluorescent levels of a marker of formula I of the present invention in the retina of a subject comprising at least (i) a light source configured to emit light to illuminate the retina of said subject, the light having a wavelength comprised between 350 and 650 nm, and (ii) an optical unit configured to detect and/or quantify the fluorescence emitted by said fluorescent marker, upon illumination of the retina with the light source, wherein said device is configured to implement any of the methods described herein.
The use of the device according to the present invention for the implementation of any of the methods as described in the present specification and in the claims.

Additional advantages, as well as the features and the use modes of the present invention will be evident from the following detailed description of some preferred embodiments, shown purely by way of example.

### Glossary

BODIPY in the present invention has the meaning commonly intended in the art, i.e. is the technical common name of a chemical compound with formula C₉H₇BN₂F₂, whose molecule consists of a boron difluoride group BF₂ joined to a dipyrromethene group C₉H₇N₂; specifically, the compound 4,4-difluoro-4-bora-3a,4a-diaza-s-indacene in the IUPAC nomenclature. The common name is an abbreviation for “boron-dipyrromethene”.

The terms aromatic and not aromatic are as commonly understood in the state of the art and can therefore indicate, respectively, any aromatic heterocycle and any non-aromatic heterocycle.

As used herein, the term “ocular tissue” can include any tissue of an eye and/or the optic nerve of a mammal, such as a retina or substructures within the retina perimeter such as macula lutea and fovea. The retina can include one or more of: an inner nuclear layer of the retina and a retinal ganglion cell of the retina.

TAU1 probe is the structure described in the manuscript by Verwilst P, et. al, “Rational Design of in Vivo Tau Tangle-Selective Near-Infrared Fluorophores: Expanding the BODIPY Universe.” J Am Chem Soc. 2017 Sep. 27; 139(38):13393-13403.

### BRIEF DESCRIPTION OF THE DRAWINGS

FIG. 1. General formula of the compound of formula I.

FIG. 2. Binding pose of BT1 compound into the Tau fibrillar cavity.

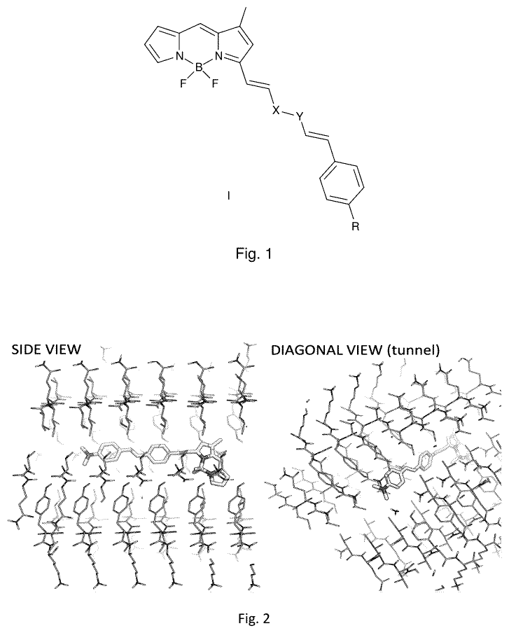

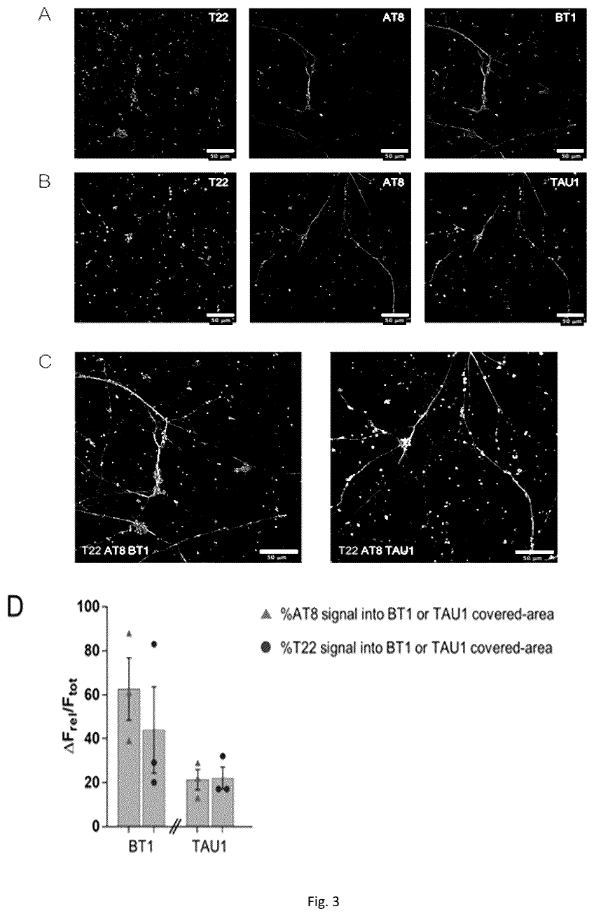

FIG. 3. (A) Representative binarized fluorescence image showing human iPSC-derived cortical neurons at 30 days in vitro after incubation with BT1 (100 μM) for 30 minutes at 37° C. and staining with anti-T22 and anti-AT8 antibodies.
(B) Representative binarized fluorescence image showing human iPSC-derived cortical neurons at 30 days in vitro after incubation with TAU1 (100 μM) for 30 minutes at 37° C. and staining with anti-T22 and anti-AT8 antibodies.
(C) Fluorescence composite of the above representative images. The fluorescent image of BT1 was obtained using excitation at 520 nm with a 555/15 nm filter in detection; TAU1 was obtained using excitation at 470 nm with a 510/5 nm filter in detection. Both probes are displayed. Cell nuclei were stained with DAPI. (D) The probe specificity is calculated as the ratio between the antibody intensity within the area covered by BT1 or TAU1 out of the total antibody intensity (n=3), as determined using Image J. Images were acquired at the FV10i confocal microscope using a BOX magnification.

FIG. 4. (A) Representative binarized fluorescence image showing human iPSC-derived retinal ganglion cells at 30 days in vitro after incubation with BT1 (100 μM) for 30 minutes at 37° C. and staining with anti-T22 and anti-AT8 antibodies.
(B) Representative binarized fluorescence image showing human iPSC-derived retinal ganglion cells at 30 days in vitro after incubation with TAU1 (100 μM) for 30 minutes at 37° C. and staining with anti-T22 and anti-AT8 antibodies.
(C) Fluorescence composite of the above representative images. The fluorescent image of BT1 was obtained using excitation at 520 nm with a 555/15 nm filter in detection; TAU1 was obtained using excitation at 470 nm with a 510/5 nm filter in detection. Both probes are displayed. Cell nuclei were stained with DAPI.
(D) The probe specificity is calculated as the ratio between the antibody intensity within the area covered by BT1 or TAU1 out of the total antibody intensity (n=3), as determined using Image J. Images were acquired at the FV10i confocal microscope using a 60× magnification.

### DETAILED DESCRIPTION

The authors of the present invention have identified, among BIODIPY derived molecules, a new fluorescent marker selectively binding Tau protein of formula I:

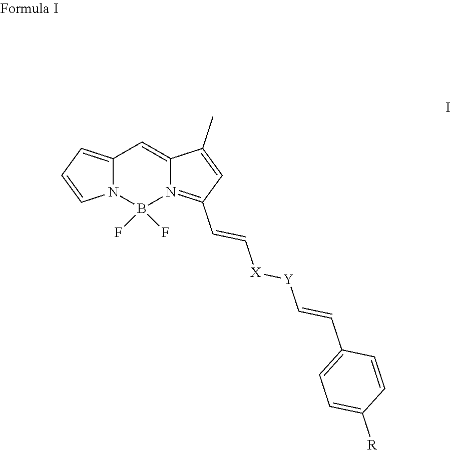

wherein X and Y are carbon atoms linked either by a double bond with E or Z configuration or by an heteroaromatic or aromatic para-substituted ring, or by an aromatic or heteroaromatic 1,4 disubstituted ring; R is hydrogen, halogen, NH(RA), N(RA)₂, NHC(═O)RA, ORA, OC(═O)RA, SRA, SO₂RA, SO₃RA, OSO₂RA, OSO₃RA, C(RA)₃, or C₅₋₇ aromatic or aliphatic heterocycle; and
in each substituent, RA is: hydrogen; halogen; hydroxyl; CF₃, a C₁₋₇ saturated or unsaturated chain, linear or branched containing up to three independent heteroatoms selected among nitrogen oxygen and sulphur; C₅₋₇ cycloalkyl; phenyl; C₅₋₇ heterocycle or n-ethyleneglycol.

The compound of formula I is a fluorescent molecule with a high affinity for Tau protein neurofibrillary tangles (NFTs).

The fluorescence marker of formula I of the present invention is a lipophilic compound demonstrating efficient penetration of the blood-brain barrier.

One embodiment of the invention is related to a fluorescent marker of formula I as defined above, wherein X and Y are carbon atoms linked by an aromatic para-substituted ring or by an aromatic 1,4 disubstituted ring;
R is H, halogen, NH(RA), N(RA)₂, NHC(═O)RA, ORA, OC(═O)RA, SRA, SO₂RA, SO₃RA, OSO₂RA, OSO₃RA, C(RA)₃, or C₅₋₇ aromatic or aliphatic heterocycle; and
in each substituent, RA is: hydrogen; halogen; hydroxyl; CF₃, a C₁₋₇ saturated or unsaturated chain, linear or branched containing up to three independent heteroatoms selected among nitrogen oxygen and sulphur; C₅₋₇ cycloalkyl; phenyl; C₅₋₇ heterocycle or n-ethyleneglycol.

According to the present invention, preferred aromatic heterocycles are selected among pyrrole, imidazole and piperidine.

According to the present invention, preferred non-aromatic heterocycles are selected among morpholine, piperazole and pyrrolidine.

In one preferred embodiment, R is selected from NH₂, NH(CH₃), N(CH₃)₂, N(Ph)₂, imidazole, morpholine, piperazine.

Non-limiting examples of fluorescent markers of formula I include 3-((E)-4-((E)-4-(dimethylamino)styryl)styryl)-5,5-difluoro-1-methyl-5H-dipyrrolo[1,2-c:2′,1′-f][1,3,2]diazaborinin-4-ium-5-uide (also referred as BT1 in the present description, in the figures and in the schemes);
3-((E)-4-((E)-4-(diphenylamino)styryl)styryl)-5,5-difluoro-1-methyl-5H-dipyrrolo[1,2-c:2′,1′-f][1,3,2]diazaborinin-4-ium-5-uide (also referred as BT2 in the present description and in the schemes);
3-((E)-4-((E)-4-aminostyryl)styryl)-5,5-difluoro-1-methyl-5H-dipyrrolo[1,2-c:2′,1′-f][1,3,2]diazaborinin-4-ium-5-uide (also referred as BT3 in the present description and in the schemes);
5,5-difluoro-1-methyl-3-((E)-4-((E)-4-(methylamino)styryl)styryl-5H-dipyrrolo[1,2-c:2′,1′-f][1,3,2]diazaborinin-4-ium-5-uide (also referred as BT4 in the present description and in the schemes);
5,5-difluoro-1-methyl-3-((1E,3E,5E)-6-(pyrrolidin-1-yl)hexa-1,3,5-trien-1-yl)-5H-dipyrrolo[1,2-c:2′,1′-f][1,3,2]diazaborinin-4-ium-5-uide (also referred as BT5 in the present description and in the schemes);
5,5-difluoro-1-methyl-3-((E)-4-((E)-4-morpholinostyryl)styryl)-5H-dipyrrolo[1,2-c:2′,1′-f][1,3,2]diazaborinin-4-ium-5-uide (also referred as BT6 in the present description and in the schemes);
5,5-difluoro-1-methyl-3-((E)-4-((E)-4-(piperazin-1-yl)styryl)styryl)-5H-dipyrrolo[1,2-c:2′,1′-f][1,3,2]diazaborinin-4-ium-5-uide (also referred as BT7 in the present description and in the schemes);
and 3-((E)-4-((E)-4-(1H-imidazol-1-yl)styryl)styryl)-5,5-difluoro-1-methyl-5H-dipyrrolo[1,2-c:2′,1′-f][1,3,2]diazaborinin-4-ium-5-uide (also referred as BT8 in the present description and in the schemes).

Therefore, the fluorescent marker of formula I can be one of:
3-((E)-4-((E)-4-(dimethylamino)styryl)styryl)-5,5-difluoro-1-methyl-5H-dipyrrolo[1,2-c:2′,1′-f][1,3,2]diazaborinin-4-ium-5-uide (BT1);
3-((E)-4-((E)-4-(diphenylamino)styryl)styryl)-5,5-difluoro-1-methyl-5H-dipyrrolo[1,2-c:2′,1′-f][1,3,2]diazaborinin-4-ium-5-uide (BT2);
3-((E)-4-((E)-4-aminostyryl)styryl)-5,5-difluoro-1-methyl-5H-dipyrrolo[1,2-c:2′,1′-f][1,3,2]diazaborinin-4-ium-5-uide (BT3);
5,5-difluoro-1-methyl-3-((E)-4-((E)-4-(methylamino)styryl)styryl)-5H-dipyrrolo[1,2-c:2′,1′-f][1,3,2]diazaborinin-4-ium-5-uide (BT4);
5,5-difluoro-1-methyl-3-((1E,3E,5E)-6-(pyrrolidin-1-yl)hexa-1,3,5-trien-1-yl)-5H-dipyrrolo[1,2-c:2′,1′-f][1,3,2]diazaborinin-4-ium-5-uide (BT5)
5,5-difluoro-1-methyl-3-((E)-4-((E)-4-morpholinostyryl)styryl)-5H-dipyrrolo[1,2-c:2′,1′-f][1,3,2]diazaborinin-4-ium-5-uide (BT6);
5,5-difluoro-1-methyl-3-((E)-4-((E)-4-(piperazin-1-yl)styryl)styryl)-5H-dipyrrolo[1,2-c:2′,1′-f][1,3,2]diazaborinin-4-ium-5-uide (BT7);
3-((E)-4-((E)-4-(1H-imidazol-1-yl)styryl)styryl)-5,5-difluoro-1-methyl-5H-dipyrrolo[1,2-c:2′,1′-f][1,3,2]diazaborinin-4-ium-5-uide (BT8).

The chemical structures of the fluorescent markers BT1-BT8 are shown in the Scheme 1 below.

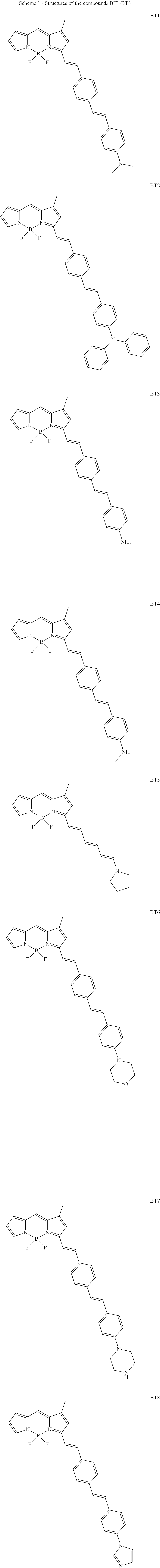

In a preferred embodiment the fluorescent marker is:
3-((E)-4-((E)-4-(dimethylamino)styryl)styryl)-5,5-difluoro-1-methyl-5H-dipyrrolo[1,2-c:2′,1′-f][1,3,2]diazaborinin-4-ium-5-uide.

The fluorescent markers of the invention are characterized by an excitation wavelength of 350 to 650 nm and an emission wavelength of 450 to 800 nm.

The invention further provides a method for the preparation of a fluorescent marker of formula I as previously defined comprising the following steps:
i. subjecting 4,4-Difluoro-1,3-dimethyl-4-bora-3a,4a-diaza-s-indacene to Knoevenagel condensation reaction with an aldehyde of formula II,

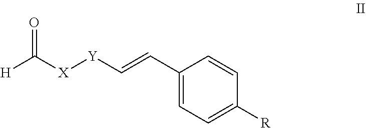

wherein X and Y are carbon atoms linked either by a double bond with E or Z configuration or by an heteroaromatic or aromatic para-substituted ring, or by an aromatic or heteroaromatic 1,4 disubstituted ring;
   R is hydrogen, halogen, NH(RA), N(RA)₂, NHC(═O)RA, ORA, OC(═O)RA, SRA, SO₂RA, SO₃RA, OSO₂RA, OSO₃RA, C(RA)₃, or C₅₋₇ aromatic or aliphatic heterocycle; and in each substituent, RA is: hydrogen, halogen hydroxyl CF₃, a C₁₋₇ saturated or unsaturated chain, linear or branched containing up to three independent heteroatoms selected among nitrogen oxygen and sulphur; C₅₋₇ cycloalkyl; phenyl; C₅₋₇ heterocycle or n-ethyleneglycol;
ii. subjecting the solution obtained in step i. to liquid-liquid extraction (LLE) followed by a purification step to provide said fluorescent marker of formula I.

In one embodiment, said Knoevenagel reaction is carried out under Dean-Stark conditions in the presence of piperidine (or pyrrolidine) and acetic acid, using toluene (or benzene or trifluoromethyl benzene) as solvent. The Knoevenagel reaction can be carried out at at reflux for 2-4 hours.

In one specific embodiment of the present invention, step ii. of the above method comprises the following steps:
ii.a adding a saturated aqueous solution of ammonium chloride (NH₄Cl) to the solution obtained in step i;
ii.b subjecting the mixture obtained in step ii.a to liquid-liquid extraction (LLE);
ii.c separating the aqueous phase obtained with step ii.b;
ii.d collecting the organic phase(s) obtained with step ii.b and dehydrating over Na₂SO₄.

Said liquid-liquid extraction (LLE) can be carried out using DCM (dichloromethane) as organic solvent.

In accordance with an embodiment of the invention, the purification step to provide said fluorescent marker of formula I is carried out by chromatography. In one specific embodiment, said purification can be performed by flash chromatography using the following eluent mixture: hexane:ethyl acetate in 9:1 ratio.

In one preferred embodiment of the above method, said aldehyde of formula II is the trans-4-[2-(4-dimethylaminophenyl) vinyl]benzaldehyde.

Said trans-4-[2-(4-dimethylaminophenyl) vinyl]benzaldehyde may be obtained by subjecting 4-bromobenzaldehyde to Heck reaction with 4-dimethylaminostyrene in the presence of a suitable catalyst and potassium carbonate.

A suitable catalyst that can be used to produce said trans-4-[2-(4-dimethylaminophenyl) vinyl]benzaldehyde starting from 4-bromobenzaldehyde and 4-dimethylaminostyrene is, for example, a catalyst prepared in situ by mixing palladium acetate (II) (Pd(CH₃COO)₂) with triphenylphosphine (PPh₃) in dimethylformamide (DMF).

The invention also relates to a composition comprising at least one fluorescent marker of the invention and optionally one or more carriers and/or excipients.

A suitable carrier and/or excipient is, for example, a solvent, such as DMSO, preferably a pharmaceutical acceptable solvent, provided that it allows to solve or stably disperse the fluorescent marker of formula I of the invention.

The concentration of the fluorescent marker of formula I in said composition can be adjusted depending on the type of fluorescent marker of the present invention. In one embodiment, the concentration of the fluorescent marker may be in the range from 0.5 micromolar to 50 millimolar. The excipient to be contained in said composition can be adjusted such that the desired dosage of the fluorescent marker is obtained.

The composition of the invention is preferably in the form of an oral composition or in the form of an ophthalmic composition.

The oral composition can be in the form of a solution, a suspension, a gel, a soft or hard gelatine, a capsule, a tablet, a lozenge, a powder, a granulate, a pill, an oleogel.

Any suitable carrier or excipient known in the art for the preparation of compositions suitable for oral administration can be used by the skilled person.

The ophthalmic composition according to the invention can be in the form of eye drops, ophthalmic ointment or ophthalmic lotion

Any suitable carrier or excipient known in the art for the preparation of compositions suitable ophthalmic compositions can be used by the skilled person.

One aspect of the present invention is referred to a fluorescent marker of formula I or to a composition comprising said fluorescent marker as previously described, for use in the detection of neurofibrillary tangles of the Tau protein.

In an embodiment, the invention is referred to a fluorescent marker of formula I or to a composition comprising said fluorescent marker as previously described, for use in the detection of neurofibrillary tangles of the Tau protein in a subject, in other words the invention is also referred to a fluorescent marker of formula I or to a composition comprising said fluorescent marker as previously described, for use in the detection of neurofibrillary tangles of the Tau protein in a subject in vivo.

In accordance with an embodiment of the invention, any of the fluorescence markers or compositions taught herein can be administered to a subject in need thereof for use in the detection of neurofibrillary tangles of the Tau protein. As stated above, said detection can be in a subject, and it can be carried out in vivo without invasive tools.

In a further embodiment of the invention the detection can be carried out in vitro on suitable samples, including organoids.

Notably, the invention further provides a method for the detection of neurofibrillary tangles of the Tau protein comprising the steps of:
contacting a fluorescent marker of formula I or a composition comprising the fluorescent marker of formula I as previously defined with a biological sample under conditions wherein said fluorescent marker binds to the neurofibrillary tangles of the tau protein;
detecting said fluorescent marker bound to the biological sample.

Non-limiting examples of biological samples that can be analysed by using the above method include an ocular tissue, a brain tissue or olfactive epithelia. In one preferred embodiment, said biological sample is an ocular tissue.

In one embodiment, the contacting step of the above method can be carried out at a temperature of 37° C. Said contacting step has a duration comprised between 10 and 120 minutes so that the fluorescent marker selectively binds the neurofibrillary tangles of the tau proteins present in the biological sample to be analysed.

The period of incubating the biological sample in the presence of said fluorescent marker depends on the amount of the fluorescent marker applied but falls within the above-mentioned range. In one embodiment, the contacting time is 30 minutes.

Preferably, in said contacting step, the fluorescent marker of the invention is in a concentration ranging from 0.5 micromolar to 50 millimolar.

The above method may further comprise, after said contacting step, a washing step in which any excess of said fluorescence marker is removed from the biological sample.

In one embodiment, the presence and/or amount of any of the fluorescent markers of the invention bound to the neurofibrillary tangles of the Tau protein within a biological sample can be determined by fluorescence measurements, preferably by fluorescence imaging.

Fluorescence imaging can be performed according to any of the fluorescence imaging techniques known in the art. For example, a qualitative and/or quantitative evaluation of the binding of the fluorescent marker of the invention to the neurofibrillary tangles of the tau protein within said biological sample can be accomplished using microscopy techniques.

In some embodiments, the detection step of the method of the present invention as described above further comprises the following steps:
illuminating said biological sample with a light source, the light source having a wavelength (A) appropriate to determine the emission of fluorescence from the bound fluorescent marker; and
detecting and optionally quantifying the fluorescence emitted by said fluorescent marker, wherein said emitted fluorescence has a wavelength in the range of from about 450 and 800 nm.

It is preferable that the excitation light source has a narrow emission range so as to avoid the excitation of any other constituents of the biological sample to be analysed. In one embodiment, the light source has a wavelength in the range of from about 350 to 650 nm, preferably is equal to 559 nm.

Yet another embodiment of the present invention is directed to an imaging method comprising the steps of:
administering to a subject a fluorescent marker of formula I or a composition comprising said fluorescent marker as defined in the present specification,
carrying out a non-invasive fluorescence imaging of the retina of said subject, wherein the detection of fluorescence from said fluorescent marker indicates the binding of said marker to the retina.

In one embodiment, the fluorescent marker of formula I or the composition comprising said fluorescent marker of formula I according to the present invention can be administered to a subject by oral administration or by ophthalmic administration.

In a preferred embodiment, any of the fluorescence markers or compositions comprising said fluorescence markers taught herein is administered to said subject from at least 30 minutes to one day prior to the fluorescence measurement. By way of example, the administration is at least 1 hour, 2 hours, at least 4 hours, at least 8 hours, ably at least 12 hours, at least 16 hours prior to the fluorescence measurement. Thanks to its capability to efficiently penetrate the blood-brain barrier, the fluorescence marker of formula I of the invention is capable of reaching the retina following administration and to selectively bind the neurofibrillary tangles of the tau protein present therein.

The time needed for said binding varies depending on the method selected for the administration of the marker or of the composition. A topical administration, in the form of an ophthalmic administration will need a shorter time for the subsequent fluorescence detection stem compared to an oral administration.

In one embodiment, the fluorescent marker of the invention is administered in an amount comprised between 0.5 micromolar to 50 millimolar per unit dosage.

Fluorescence imaging of the retina of a subject according to the methods of the present invention can be performed by way of any fluorescence imaging technique known in the art, as long as it is non-invasive. Non-limiting examples of non-invasive fluorescence imaging techniques include scanning laser ophthalmoscopy (SLO), confocal scanning laser ophthalmoscopy (cSLO), or fluorescence lifetime imaging ophthalmoscopy (FLIO). The fluorescence imaging can be qualitative or quantitative, in other terms it can indicate merely the presence or absence of fluorescence or it can quantify the amount of fluorescence detected.

By way of example, non-invasive fluorescence imaging of the retina can be carried out by employing a light source to illuminate the retina of said subject, together with means for detecting and/or quantifying the fluorescence emitted by the fluorescence marker of the invention bound to the neurofibrillary tangles of the tau protein within the retina, wherein said emitted fluorescence has an emission wavelength in the range of from about 450 to 800 nm.

As used herein, a “light source” may be any light source that can be configured to illuminate the retina of a subject, having a wavelength appropriate to determine the emission of fluorescence from the bound fluorescent marker of the invention within the retina of said subject. In one preferred embodiment, said light source has a wavelength comprised between 350 to 650 nm, preferably equal to 559 nm.

In accordance with an embodiment of the invention, detecting and/or quantifying the fluorescence emitted by the bound fluorescence marker of the invention within the retina can be performed by means of any suitable device configured for fluorescence detection and recording. An example of suitable device is a device comprising a unit configured to receive the fluorescence produced as a result of the illumination of the retina of said subject and to detect the fluorescence emitted by the fluorescent marker of formula I bound to the neurofibrillary tangles of the tau protein within the retina, namely permitting to distinguish the presence and/or to quantify the amount of said fluorescent marker bound to the retina. The fluorescent marker of the invention will be detectable only when bound to the retina as the amount trapped in the tangles will be sufficient to emit sufficient fluorescence for the detection thereof. Unbound, freely diffusing marker, due to its low water solubility is not likely to contribute to the observed fluorescent signal.

In one embodiment, the device may comprise a camera configured to form a camera image of the fluorescence emitted by the marker bound to the retina to be illuminated with the appropriate light source.

Such device may be programmed to analyse the collected fluorescence intensity and to provide a quantitative measurement of the fluorescent marker of the invention within the retina, for example by calculating an average fluorescence intensity value of said bound marker, through the use of a dedicated computer program and/or any suitable software available to the public.

Hence, in one embodiment of the invention, the above imaging method may comprise determining the peak intensity of fluorescence produced by the fluorescent marker of formula I bound to the NFTs of the tau protein within the retina. The amount of the fluorescent marker bound to the NFTs of the tau protein may be determined based on said peak intensity.

By way of example, the fluorescent markers of formula I of the present invention bound to the NFTs of the tau protein within the retina may be excited by picosecond laser pulses and the fluorescence emission can be detected using time correlated single photon counting (TCSPC) technology.

Advantageously, images obtained with the detection of fluorescence of the retina of a subject with the imaging method of the invention, can be used for the diagnosis of tauopathies. As neurofibrillary tau tangles are associated to tauopathies, the detection of neurofibrillary tangles in the retina can be used for the diagnosis or to assist in the diagnosis of tauopathies.

As used herein, the term “tauopathy” encompasses the class of neurodegenerative diseases involving the aggregation of tau proteins into neurofibrillary or glialfibrillary tangles (NFTs), such as Alzheimer's disease, Down syndrome, amyotrophic lateral sclerosis, Pick's disease, Parkinson's disease, primary age-related tauopathy (PART), chronic traumatic encephalopathy (CTE), progressive supranuclear palsy (PSP), corticobasal degeneration (CDB), frontotemporal dementia and parkinsonism linked to chromosome 17 (FTDP-17), lytico-bodig disease (Parkinson-dementia complex of Guam), ganglioglioma and gangliocytoma, meningiomatosis, postencephalitic parkinsonism, subacute sclerosing panencephalitis (SSPE), as well as lead encephalopathy, tuberous sclerosis, Pantothenate kinase-associated neurodegeneration, and lipofuscinosis.

In one preferred embodiment, said tauopathy is selected from Alzheimer's disease, Down syndrome, amyotrophic lateral sclerosis, Pick's disease, or Parkinson's disease, preferably is Alzheimer's disease.

In accordance with an embodiment of the invention, the method for detecting neurofibrillary tangles of tau protein in a biological sample or any of the imaging methods as described in the present specification can be combined with results obtained by other imaging techniques, for example, a technique for detecting amyloid proteins, in order to aid in a diagnosis of a tauopathy.

A method for determining a fluorescence graph comprising the steps of:
administering to a subject a fluorescent marker according to any one of claims from 1 to 6 or a composition comprising said fluorescent marker as defined in claim 11 and carrying out a non-invasive quantitative fluorescence imaging on the retina of said subject, at a plurality of successive time instants tᵢ, where i is 0 to n, so obtaining corresponding fluorescence values;
using the obtained fluorescence values for determining a graph of the fluorescence as a function of time.

In implementing the method described above, the time instant tᵢ with i=0 represents the time in which monitoring is initiated. The successive time instants tᵢ in which i is an integer value increasing from 0+1 to n, are instants of time subsequent to each other, their succession in time being indicated by the increase of the i value.

Hence, the instant of time tᵢ with i=0 is the instant in which the monitoring is initiated, and the fluorescence value of the fluorescent marker of formula I in the retina at this tᵢ can be considered as the starting fluorescence value from which the progression in time of said fluorescence within the retina can be assessed.

Non-invasive quantitative fluorescence imaging of the retina can be repeated in time instants subsequent to tᵢ with i=0 and subsequent to each other in progression from i=1 to n, . . . so that the time instant t₁ precedes the time instant t₂, which precedes the time instant t₃ and so on.

Comparison of the fluorescence values in the images obtained for each subsequent tᵢ according to the above method can hence be elaborated in a graph of the fluorescence as a function of time.

A graph as the one provided by the method above, can be advantageously used to assess the effectiveness of a medical treatment against a tauopathy or to assess the progression in time of a thauopathy.

In this case, the time tᵢ with i=0 corresponds to the time at which medical treatment outcome is monitored, this can advantageously be before the start of the treatment itself but can be introduced at any time during the treatment even after the treatment has already started.

A progressive decrease or a stability of the fluorescence in the graph provided by the method above, can be indicative of the effectiveness of the medical treatment in ameliorating the tauopathy. The meaning of the graph and the related extent of the effectiveness can be evaluated by the interpretation of the graph by the clinical expert.

A progressive increase of the fluorescence in the graph provided by the method above can be indicative of the ineffectiveness of the medical treatment. The meaning of the graph and the related extent of the ineffectiveness can be evaluated by the interpretation of the graph by the clinical expert.

In some cases, changing dietary habits and lifestyle may be suggested to a subject before undergoing a medical treatment. In these cases, it may be useful to monitor the progression of the disease over time, to see, for example, if changes in dietary habits and lifestyle have a positive effect on the disease.

The graph provided by the method above can be used to monitor the effects of said changes on the disease.

Anyway, monitoring the progression of a tauopathy on a subject, regardless of whether or not a possible therapeutic efficacy is evaluated, may be of interest for a medical doctor.

Therefore, the method for determining a fluorescence graph as described in the present specification can be advantageously used for monitoring the progression of a tauopathy on a subject.

What has been previously described regarding the administration modes and dosages of the fluorescent marker of formula I or compositions comprising said fluorescent marker in the explanation of the general imaging method, also applies to any of the other methods described above.

Non-invasive quantitative fluorescence imaging of the retina of a subject to be analyzed may be performed according to any one of the non-invasive fluorescence imaging techniques known in the art, including those as previously described in the present specification. In particular, according to one preferred embodiment of the invention, in any of the above imaging methods, said fluorescence imaging is carried out by submitting the retina of said subject to irradiation with a light source having a wavelength (λ) comprised between 350 and 650 nm, and detecting and/or quantifying the fluorescence emitted by said fluorescent marker.

Any of the above methods comprising the fluorescence imaging of the retina, may further comprise “normalizing” a determined amount or the fluorescence level of the fluorescence marker of the invention bound to the NFTs of the tau protein, based on the background autofluorescence emitted from the retina of said subject. As used herein, such “normalizing” can include subtracting the amount of background autofluorescence from the amount of fluorescence emitted from the fluorescent marker of the invention bound to the NFTs of the tau protein; it can also include determining a ratio of such quantities; and can include using such a normalized result as a normalized measure of the amount of the fluorescent marker of formula I of the present invention bound to the NFTs of the tau protein.

The detection of neurofibrillary tangles of tau protein in the retina allows an early and non-invasive diagnosis of a tauopathy, the high specificity of the fluorescent markers of the present invention renders the detection of the tangles more sensitive and accurate, thereby improving an early diagnosis of the disease.

The imaging method of the invention thereby can be used in methods of treatment of tauopathies in which the early diagnosis of the disease is followed by appropriate treatments of the same at stages in which normally no diagnosis is possible.

In addition, the invention also encompasses a method of treatment of a tauopathy in which the effectiveness of a medical treatment is monitored by the analysis of the graph of the fluorescence as a function of time provided by the present invention and the medical treatment is continued, variated or changed by the doctor in charge depending on the outcome of said monitoring.

The following experimental section is provided solely by way of illustration and not limitation and does not intend to restrict the scope of the invention as defined in the appended claims. The claims are an integral part of the description.

### EXAMPLES

### Example 1—Design of Selective Markers for Tau Protein Neurofibrillary Tangles (NFTs) and Molecular Docking

A series of fluorescent probes have been designed, named BT1-BT8, consisting of a BODIPY core functionalized in position 3 with a highly conjugated system ending with an aliphatic amine, cyclic and non, or aromatic, characterized by a distance between the electron donor portion and the acceptor portion of 13-19 Å and by a different polarity, as shown in the scheme 2, below.

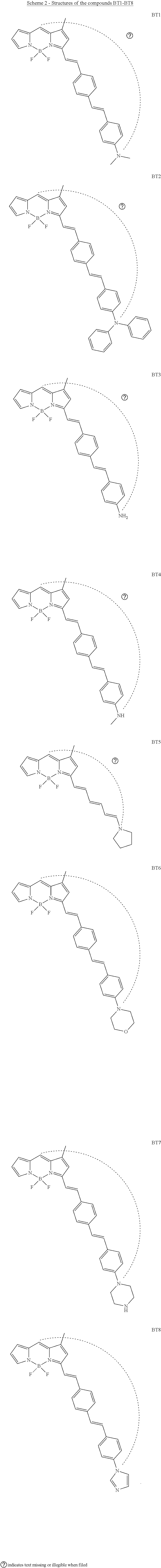

In order to assess the selectivity towards NFTs and excluding any unlike candidates, the molecules were screened in silico against the crystallographic structure of the PHF6 fragment responsible for the propensity of the protein itself to assemble into fibrils. The 6-mer model of the hexapeptide 306VQIVYK311 of the PHF6 fragment, present in the R3 region of the microtubule-binding tau protein, was built using elongation and symmetry operators, in accordance with the procedure reported in the literature. [Verwilst P. et al., 2017]

The high-resolution crystallographic structure of the peptide, coded by PDB-ID 5K7N. [de la Cruz M. J. et al, 2017] was used as a model system to build 6-Wed. The docking of small molecules was carried out inside the preserved and amphiphilic tunnel formed by the peptide monomers. Molecular docking was performed with AutoDock4.2 [Morris G. M. et al, 2009]

The ligands were drawn in Picto (OpenEye) and subsequently converted into three-dimensional format using OMEGA (OpenEye). [Hawkins PCD et al., 2010] It should be noted that the OpenEye and AutoDock4.2 softwares do not provide force field parameters for docking compounds containing boron, for this reason the boron atom has been replaced with a carbon atom hybridized sp3. The ionization state of the pH 7.4 molecules was assessed with QUACPAC (OpenEye) (QUACPAC 2.0.2.2: OpenEye Scientific Software, Santa Fe, NM. Http://www.eyesopen.com) while the specific format PDBQT compatible with AutoDock it was generated with the AutoDockTool GUI. [Morris G. M. et al, 2009]

For each ligand 10 runs of the genetic algorithm were performed, the statistically most relevant docking poses were determined through a combined analysis of the scores and visual inspection.

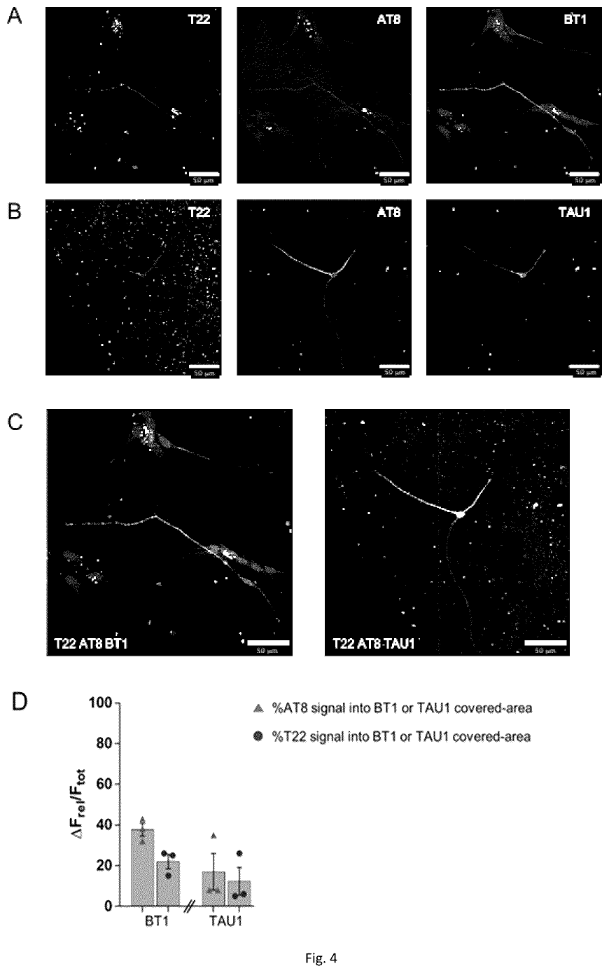

The binding pose of BT1 compound into the Tau fibrillar cavity is shown in FIG. 2.

The Table 1 below summarizes the binding affinities as predicted for compounds BT1-BT8.

TABLE 1
Binding affinities as predicted for compounds BT1-BT8.

| MARKER | Estimated free energy of binding (kcal/mol) | |
|---|---|---|
| BT1 | −9.71 | |
| BT2 | −11.60 | (POSE 1) |
| | −10.39 | (POSE 2) |
| BT3 | −9.38 | |
| BT4 | −9.75 | |
| BT5 | −3.99 | |
| BT6 | −7.95 | |
| BT7 | −5.72 | |
| BT8 | −7.62 | (POSE 1) |
| | −2.51 | (POSE 2) |

The BT1 compound was found to be the most promising compound as a selective marker of the Tau protein NFTs in terms of in silico affinity, binding conformation and polarity.

### Example 2—Design and Synthesis of the BT1 Compound

In brief, for the synthesis of the BT1 compound, a synthetic two-step strategy was developed: Knoevenagel condensation between the selected and commercially available Bodipy core and the trans-4-[2-(4-dimethylaminophenyl) vinyl]benzaldehyde; the latter was synthesized by Heck reaction between 4-bromobenzaldehyde and 4-dimethylaminostyrene, both commercially available, in the presence of a catalyst suitably chosen to promote the stereoselectivity of the reaction (as shown in Scheme 3).

### Chemicals, Reagents and Methods of Analysis

All reagents and solvents are available on the market and have been used without further purification.

Silica gel (230-400 mesh) was used for purification by column flash chromatography. All reactions were monitored by thin layer chromatography (TLC) and f254 fluorescence gel silica plates (Sigma-Aldrich 99569) were used. Melting points were determined with Melting Point B-454 apparatus. The 1H and 13C NMR spectra were recorded with a Bruker 400 Ultra Shield™ instrument (400 MHz for 1H NMR and 100 MHz for 13C NMR), using tetramethylsylene (TMS) as standard. Chemical displacements are reported in parts for millions (ppm). The multiplicity has been reported as follows: singlet (s), doublet (d), triplet (t) and multiplex (m). Mass spectrometry was performed with the Thermo Finnigan LXQ linear ion trap mass spectrometer, equipped with electrospray ionization (ESI). High-resolution mass spectra (HR-MS) were recorded with a Bruker BioApex Fourier transform ion cyclotron resonance (FT-ICR).

### Synthetic Procedures

The trans-4-[2-(4-dimethylminophenyl)vinyl]benzaldehyde (2) compound was prepared by the Heck reaction as shown in the following Scheme 3:

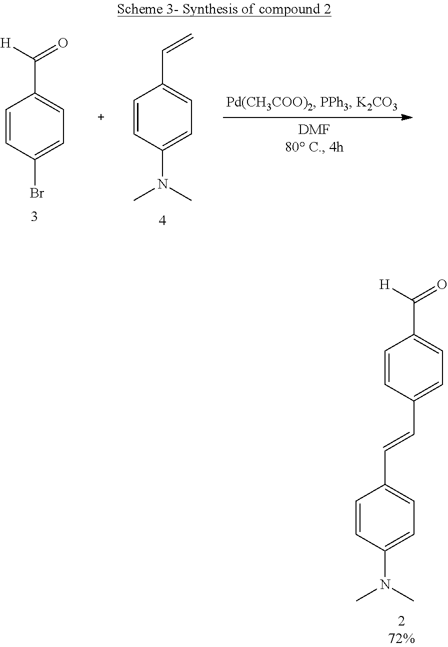 [SMILES: O=Cc1ccc(Br)cc1 ; C=Cc1ccc(N(C)C)cc1 ; CN(C)c1ccc(/C=C/c2ccc(C=O)cc2)cc1]

The catalyst was prepared in situ: palladium acetate (II) Pd(CH3COO)2)(Merck Life Science 3375-31-3)(16.8 mg, 0.075 mmol) and triphenylphosphine (PPh3)(Merck Life Science 603-35-0) (19.7 mg, 0.075 mmol) have been soluble in dimethylformamide (DMF) (Merck Life Science 6812-2). After 10 minutes, a solution of 4-bromobenzaldehyde 3 (Merck Life Science 1122-91-4) (202 mg, 1.5 mmol, 4-dimethylminostyrene 4 (Building Block, Merck Life Science 2039-80-70) (264.6 mg, 1.8 mmol) and potassium carbonate (K2CO3) (Merck Life Science 584-08-7) (414 mg, 3.00 mmol) in DMF (3 m) has been added to the catalyst solution. The reaction was left in agitation at 80° C. for 4 h. Later, the reaction was extracted with CH2Cl2 (3 times) and the organic phases were joined, dehydrated with Na2SO4 anhydrous and concentrated at reduced pressure. The trans-4-[2-(4-dimethylminophenyl)vinyl]benzaldehyde 2 compound (1,074 mmol, 270 mg) was obtained with a yield of 72% by cold hexane crystallization.

Yellow solid (yield 72%). mp: 218.0-220.0° C.

¹H NMR (400 MHz, CDCl₃) δ 9.96 (s, 1H), 7.83 (d, J=8.2 Hz, 2H), 7.60 (d, J=8.2 Hz, 2H), 7.45 (d, J=8.7 Hz, 2H), 7.21 (d, J=16.2 Hz, 1H), 6.94 (d, J=16.2 Hz, 1H), 6.72 (d, J=8.7 Hz, 2H), 3.01 (s, 5H). ¹³C NMR (101 MHz, CDCl₃) δ 191.75, 150.77, 144.69, 134.62, 132.66, 130.40, 128.33, 126.38, 124.86, 122.82, 112.40, 40.47. ESI-MS (m/z): [M+H]⁺ calcd. for C₁₇H₁₈NO, 252.13; found, 252.17.

The compound BT1 has been prepared by a Knoevenagel condensation (Scheme 4 below).

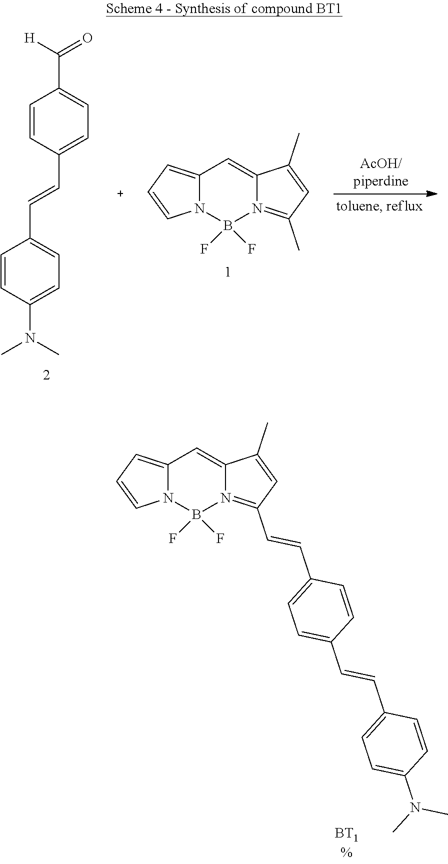

A solution of 4,4-Difluoro-1,3-dimethyl-4-bora-3a,4a-diaza-s-indicene (TCI Europe 154793-49-4) (100 mg, 0.45 mmol) and trans-4-[2-(4-dimethylminophenyl) vinyl]benzaldehyde (112.95 mg, 0.45 mmol), in the presence of piperidine (Merck Life Science 110-89-4) (0.35 ml, 6.12 mmol) and acetic acid (Merck Life Science 64-19-7) (0.35 ml, 3.5 mmol, in 10 ml of Toluene (Merck Life Science 108-88-3) was distilled at 120° C. for 2-4 hours. Later, the Dean-Stark was removed. The reaction was brought to room temperature and 50 ml of an aqueous solution of NH₄Cl ammonium chloride was added. Subsequently, the aqueous phase was extracted with CH₂Cl₂ (3×50 m) and the organic phases were joined, dehydrated with Na₂SO₄ anhydrous and concentrated at reduced pressure. Reaction raw was purified by flash chromatography using an exane eluent mixture:ethyl acetate in a 9:1 ratio. Compound 3-((E)-4-((E)-4-(dimethylamino) styryl)styryl)-5,5-difluoro-1-methyl-5H-dipyrrol[1,2-c:2′,1′-f][1,3,2]diazaborinin-4-ium-5-uide BT1 (mmol, mg) was obtained with a yield of 33%.

Black solid (yield 33%). mp: 257-262° C.

¹H NMR (400 MHz, CD₂Cl₂) δ 7.64-7.56 (m, 4H), 7.53 (d, J=8.2 Hz, 2H), 7.47-7.41 (m, J=8.2 Hz, 3H), 7.23 (s, 1H), 7.16 (d, J=16.2 Hz, 1H), 6.94 (d, J=16.2 Hz, 1H), 6.83 (s, 1H), 6.72 (d, J=8.2 Hz, 2H), 6.49-6.46 (m, 1H), 2.99 (s, 6H), 2.34 (s, 3H).

¹³C NMR (101 MHz, CDCl₃) δ 159.68, 140.93, 140.73, 138.75, 134.63, 133.69, 131.02, 130.30, 129.00, 128.41, 126.96, 126.12, 125.63, 123.60, 123.36, 118.80, 117.96, 117.84, 117.42, 116.77, 112.80, 40.68, 30.26. ESI-MS (m/z): [M+H]⁺ calcd. for C₁₈H₂₆BF₂N₃, 453.22; found, 454.33.

### Example 3—In Vitro Tests

Human iPSCs Maintenance

Human induced-pluripotent stem cells (hiPSCs) were maintained by clonal propagation in mTeSR Plus medium (STEMCELL Technologies) on growth factor-reduced Matrigel-coated (Corning; dilution 1:100) plates at 37° C. in 5% CO2.

Human iPSCs Differentiation to Retinal Ganglion Cells

The differentiation protocol was a revisited version of Sluch V. et al, 2017 with minor modifications. Human iPSCs were dissociated to single cells with 1× Accutase (Merck Life science) and plated on growth factor-reduced Matrigel coated plates at a density of 1000 cells/mm2 in mTeSR Plus supplemented with 10 μM Rock-inhibitor Y-27632 (Peprotech). The day of seeding was designated as day minus 2 (D−2).

The day after (D−1), the stem cell medium was completely replaced with neurogenic basal medium (N2B27) consisting of 50% DMEM/F12 [1:1], 50% Neurobasal with 1% GlutaMAX, 1% non-essential amino acid (NEAA), 1% N2 Supplement, and 2% B27 Supplement without vitamin A (all from ThermoFisher Scientific). After more 24 hours (D0), fresh N2B27 medium was supplemented with a mix of small molecules consisting of 25 μM Forskolin (Peprotech), 1 μM Dorsomorphin (Peprotech), 2.5 μM IDE2 (Peprotech) and 10 mM Nicotinamide (Peprotech). At this stage, the culture medium was daily replaced in order to enhance the stem cell commitment towards an anterior ventral forebrain.

About a week after seeding (D7), a uniform confluent neuroepithelium-like sheet was visible, and cells were dissociated with 1× Accutase and plated onto poly-L-Omithine/Laminin-coated (Merck Life science) dishes at density of 1000/mm2 in N2B27 plus 10 μM Rock Inhibitor. The day after the medium was switched to N2B27 with 25 μM Forskolin and 10 mM Nicotinamide and changed every day for the next 3-4 days. Thereafter, N2B27 was supplemented only with 25 μM Forskolin, 10 ng/mL IGF1 (Peprotech) and 10 ng/mL FGF2 (Peprotech) and refreshed twice a week to promote the retinal progenitor cell proliferation and expansion.

Reached a highly density confluent state (around at D18-20), the retinal progenitor sheet was dissociated with 1× Accutase and plated onto poly-L-Omithine/Laminin-coated dishes at density of 700 cells/mm2. To enhance the exit from a proliferative state and RGCs maturation, N2B27 medium was supplemented with 10 μM Rock inhibitor (only for the seeding), 10 μM DAPT (Peprotech), 25 μM Forskolin and the medium was replaced every 3 days until day 30-35.

Human iPSCs Differentiation to Cortical Neurons

Human iPSC-cortical neurons were differentiated with a two-step protocol based on doxycycline-induced human NGN2 gene overexpression. Briefly, human iPS cells were treated with 1× Accutase and plated onto growth factor reduced Matrigel-coated plates at a density of 1000 cells/mm2 in mTeSR Plus containing 10 μM Rock-inhibitor Y-27632. The day of seeding is set as day minus 3 (D−3). One day after seeding (D−2), the medium is switched to N2 medium consisting of DMEM/F12 [1:1], 1% N2 supplement, 1% NEAA, 1% GlutaMAX supplemented with 2 μg/mL doxycycline (Merck Life Science) to induce human NGN2 expression. N2 medium was refreshed every day. Three days after (D0), the early born neurons were dissociated with Accutase and plated onto PDL/laminin-coated dishes at a density of 500 cells/mm2 in maturation medium consisting of Neurobasal, 2% B27 with vitamin A, 1% GlutaMAX, 0.5 μg/mL laminin (Merck Life Science), 20 ng/mL BDNF (Peprotech), 20 ng/mL ascorbic acid (Peprotech), 10 ng/mL GDNF (Peprotech) supplemented with 2 μg/mL doxycycline, 10 μM Rock-inhibitor Y-27632 and 10 μM DAPT. After 24 hours, Y-27632 was removed, while DAPT and doxycycline were kept in the medium until day 5. Optionally, 5 μM Ara-C (Merck Life Science) was added to the medium from day 6 to day 10 to remove no-neuronal proliferative cells. Thereafter, the medium was half changed weekly until the experimental window was reached around D30.

Staining with BODIPY-Base Probes

Human iPSC-derived neuronal cultures were incubated with either 100 μM TAU1 probe or 100 μM BT1 probe for 30 minutes at 37° C. and then fixed for 15 minutes at room temperature with cool and fresh-made 4% PFA.

TAU1 probe is the structure described in the manuscript by Verwilst P, et. al, “Rational Design of in Vivo Tau Tangle-Selective Near-Infrared Fluorophores: Expanding the BODIPY Universe.” J Am Chem Soc. 2017 Sep. 27; 139(38):13393-13403.

### Immunocytochemistry

Fixed hiPSC-derived cortical neurons and RGCs were permeabilized with 0.2% Triton X-100 (Merck Life Science) in 1×TBS and incubated for 1 hour in blocking solution containing 1×TBS, 0.2% Triton X-100, and 5% goat serum (Merck Life Science). The cells were thus incubated in blocking solution containing primary antibody overnight at 4° C. The primary antibodies employed in this study were goat anti PHF-tau Ser202/Thr205 (AT8; dilution 1:200; Thermo Fisher Scientific) and mouse anti-oligomeric TAU (T22; dilution 1:200; Merck Life Science) followed by incubation with secondary antibody (dilution 1:1000) for 1 hours at room temperature. Images were acquired with an FV10i confocal system (Olympus) with a 60× water-immersion objective lens. Fluorescence intensity per field of view was determined using the Software Image J.

The ability of the BT1 probe to stain specifically intracellular TAU aggregates was determined as a function of the antibody fluorescent signal detected within the binarized probe signal.

FIGS. 3 and 4 show the binarized signal of T22 antibody. AT8 antibody, and BT1 or TAU1 probe detected in monolayer cultures of iPSC-derived cortical and retinal neurons after 30 days in vitro. Interestingly, the BT1 probe shows higher colocalization with AT8 signal in respect to T22, indicating that the BT1 probe preferentially stains intracellular aggregates enriched with phosphorylated TAU isoforms rather than oligomeric states of TAU.

Moreover, the BT1 probe reveals a higher ability to detect phosphorylated aggregates when compared to the TAU1 probe. Although with different performance, the enhanced detection of AT8-positive aggregates displayed by the BT1 probe is preserved between PSC-derived cortical neurons and iPSC-derived retinal ganglion cells, while the TAU1 probe's performance is comparable.

1. A fluorescent marker selectively binding Tau protein of formula I: 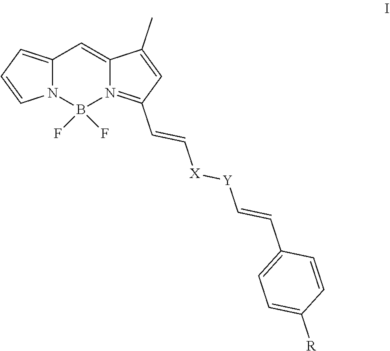
wherein X and Y are carbon atoms linked either by a double bond with E or Z configuration or by an aromatic or heteroaromatic para-substituted ring, or by an aromatic or heteroaromatic 1,4 disubstituted ring;
R is hydrogen, halogen, NH(RA), N(RA)₂, NHC(═O)RA, ORA, OC(═O)RA, SRA, SO₂RA, SO₃RA, OSO₂RA, OSO₃RA, C(RA)₃, or C₅₋₇ aromatic or aliphatic heterocycle; and
in each substituent, RA is: hydrogen; halogen; hydroxyl; CF₃; a C₁₋₇ saturated or unsaturated chain, linear or branched containing up to three independent heteroatoms selected from the group consisting of nitrogen, oxygen, and sulphur; C₅₋₇ cycloalkyl; phenyl; C₅₋₇ heterocycle; or n-ethyleneglycol.

2. The fluorescent marker according to claim 1, wherein X and Y are carbon atoms linked by an aromatic para-substituted ring or by an aromatic 1,4 disubstituted ring;
R is hydrogen, halogen, NH(RA), N(RA)₂, NHC(═O)RA, ORA, OC(═O)RA, SRA, SO₂RA, SO₃RA, OSO₂RA, OSO₃RA, C(RA)₃, or C₅₋₇ aromatic or aliphatic heterocycle; and
in each substituent, RA is: hydrogen; halogen; hydroxyl; CF₃; a C₁₋₇ saturated or unsaturated chain, linear or branched containing up to three independent heteroatoms selected from the group consisting of nitrogen, oxygen, and sulphur; C₅₋₇ cycloalkyl; phenyl; C₅₋₇ heterocycle; or n-ethyleneglycol.

3. The fluorescent marker according to claim 2, wherein R is NH₂, NH(CH₃), N(CH₃)₂, N(Ph)₂, imidazole, morpholine, or piperazine.

4. The fluorescent marker according to claim 1, wherein said fluorescent marker is selected from the group consisting of:
3-I-4-((E)-4-(dimethylamino)styryl)styryl)-5,5-difluoro-1-methyl-5H-dipyrrolo[1,2′:2′,1′-f][1,3,2]diazaborinin-4-ium-5-uide;
3-((E)-4-((E)-4-(diphenylamino)styryl)styryl)-5,5-difluoro-1-methyl-5H-dipyrrolo[1,2′:2′,1′-f][1,3,2]diazaborinin-4-ium-5-uide;
3-((E)-4-((E)-4-aminostyryl)styryl)-5,5-difluoro-1-methyl-5H-dipyrrolo[1,2′:2′,1′-f][1,3,2]diazaborinin-4-ium-5-uide;
5,5-difluoro-1-methyl-3-((E)-4-((E)-4-(methylamino)styryl)styryl)-5H-dipyrrolo[1,2′:2′,1′-f][1,3,2]diazaborinin-4-ium-5-uide;
5,5-difluoro-1-methyl-3-((1E,3E,5E)-pyrrolidinedin-1-yl)hexa-1,3,5-trien-1-yl)-5H-dipyrrolo[1,2′:2′,1′-f][1,3,2]diazaborinin-4-ium-5-uide;
5,5-difluoro-1-methyl-3-((E)-4-((E)-4-morpholinostyryl)styryl)-5H-dipyrrolo[1,2′:2′,1′-f][1,3,2]diazaborinin-4-ium-5-uide;
5,5-difluoro-1-methyl-3-((E)-4-((E)-4-(piperazin-1-yl)styryl)styryl)-5H-dipyrrolo[1,2′:2′,1′-f][1,3,2]diazaborinin-4-ium-5-uide; and
3-((E)-4-((E)-4-(1H-imidazol-1-yl)styryl)styryl)-5,5-difluoro-1-methyl-5H-dipyrrolo[1,2′:2′,1′-f][1,3,2]diazaborinin-4-ium-5-uide.

5. The fluorescent marker according to claim 4, wherein said fluorescent marker is 3-((E)-4-((E)-4-(dimethylamino)styryl)styryl)-5,5-difluoro-1-methyl-5H-dipyrrolo[1,2′:2′,1′-f][1,3,2]diazaborinin-4-ium-5-uide.

6. The fluorescent marker of claim 1, wherein said fluorescent marker has an excitation wavelength of 350 nm to 650 nm and an emission wavelength of 450 nm to 800 nm.

7. A method for preparation of a fluorescent marker according to claim 1 comprising the following steps:
i. subjecting 4,4-Difluoro-1,3-dimethyl-4-bora-3a,4a-diaza-s-indacene to Knoevenagel condensation reaction with an aldehyde of formula II, 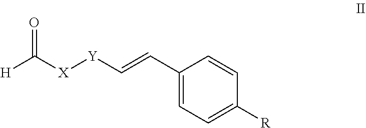
wherein X and Y are carbon atoms linked either by a double bond with E or Z configuration or by an aromatic or heteroaromatic para-substituted ring, or by an aromatic or heteroaromatic 1,4 disubstituted ring;
R is hydrogen, halogen, NH(RA), N(RA)₂, NHC(═O)RA, ORA, OC(═O)RA, SRA, SO₂RA, SO₃RA, OSO₂RA, OSO₃RA, C(RA)₃, or C₅₋₇ aromatic or aliphatic heterocycle; and in each substituent, RA is: hydrogen; halogen; hydroxyl; CF₃; a C₁₋₇ saturated or unsaturated chain, linear or branched containing up to three independent heteroatoms selected from the group consisting of nitrogen, oxygen, and sulphur; C₅₋₇ cycloalkyl; phenyl; C₅₋₇ heterocycle; or n-ethyleneglycol to obtain a solution;
ii. subjecting the solution obtained in step i. to liquid-liquid extraction (LLE); and
iii. purifying said fluorescent marker of formula I.

8. The method according to claim 7, wherein said step i. is carried out in the presence of piperidine and acetic acid.

9. The method according to claim 7, wherein said aldehyde of formula II is trans-4-[2-(4-dimethylaminophenyl) vinyl]benzaldehyde.

10. The method according to claim 7, wherein said step ii. comprises the following steps:
ii.a adding a saturated aqueous solution of ammonium chloride (NH₄Cl) to the solution obtained in step i. to obtain a mixture;
ii.b subjecting the mixture obtained in step ii.a to liquid-liquid extraction (LLE) obtaining an aqueous phase and organic phase(s);
ii.c separating the aqueous phase obtained with step ii.b;
ii.d collecting the organic phase(s) obtained with step ii.b and dehydrating over Na₂SO₄.

11. A composition comprising a fluorescent marker according to claim 1 and one or more additional excipients and/or carriers.

12. The composition of claim 11 in the form of an oral composition or of an ophthalmic composition.

13. The fluorescent marker according to claim 1 or a composition comprising said fluorescent marker and one or more additional excipients and/or carriers for detection of neurofibrillary tangles of the Tau protein.

14. A method for detection of neurofibrillary tangles of the Tau protein comprising the steps of:
contacting a fluorescent marker according to claim 1 or a composition comprising the fluorescent marker and one or more additional excipients and/or carriers, with a biological sample under conditions wherein said fluorescent marker binds to the neurofibrillary tangles of the tau protein; and
detecting said fluorescent marker bound to the biological sample.

15. An imaging method comprising the steps of:
administering to a subject a fluorescent marker according to claim 1 or a composition comprising said fluorescent marker and one or more additional excipients and/or carriers; and
carrying out a non-invasive fluorescence imaging of a retina of said subject, wherein detection of fluorescence from said fluorescent marker indicates binding of said fluorescent marker to the retina.

16. A method for determining a fluorescence graph comprising the steps of:
administering to a subject a fluorescent marker according to claim 1 or a composition comprising said fluorescent marker and one or more additional excipients and/or carriers;
carrying out a non-invasive quantitative fluorescence imaging on a retina of said subject, at a plurality of successive time instants ti, wherein i is 0 to n, so obtaining corresponding fluorescence values; and
using the obtained fluorescence values for determining a graph of fluorescence as a function of time.

17. The method of claim 16, wherein said successive time instants are separated by a time period of one or more weeks or one or more months between each time instant and a subsequent time instant.

18. The method of claim 15, wherein the non-invasive quantitative fluorescence imaging is carried out after 30 minutes to one day from said administration.

19. The method of claim 15, wherein said non-invasive quantitative fluorescence imaging is carried out by submitting the retina of said subject to irradiation with a light source having a wavelength (λ) comprised between 350 nm and 650 nm, and detecting and/or quantifying the fluorescence emitted by said fluorescent marker.

20. A method according to claim 15, wherein said fluorescence marker or said composition is administered orally or in the form of an ophthalmic ointment or eyedrops.
